(19) United States
(12) Patent Application Publication
(10) Pub. No.: US 2002/0099276 A1
(43) Pub. Date: Jul. 25, 2002

(54) Method and system for acquiring at least one measured quantity for the diagnosis or the evaluation of the pathogenesis of a central nervous system disorder of a person

(76) Inventors: Volker Schmidt, Erlangen (DE); Werner Striebel, Schwarzenbruck (DE)

Correspondence Address: SCHIFF HARDIN & WAITE, 6600 SEARS TOWER, 233 S WACKER DR, CHICAGO, IL, 60606-6473, US

(21) Appl. No.: 10/056,196

(22) Filed: Jan. 25, 2002

(30) Foreign Application Priority Data
Jan. 25, 2001 (DE) 10103328.1
Dec. 7, 2001 (DE) 10160074.7

(51) Int. Cl. A61B 5/00

(52) U.S. Cl. 600/300; 434/236

(57) ABSTRACT

In a method and a system for acquiring at least one measured quantity for diagnosis or for evaluation of the pathogenesis of a central nervous system disorder of a person while the person, on a computer, plays a game offered from a central computer via a communication network. At least one measured quantity, related to play of the game, is required for diagnosis or for evaluating the pathogenesis of a central nervous system disorder of the person. The measured quantity can be a game result that can be influenced by the person or an indicator that relates to the play behavior of the person. The measured quantity is stored in a central data bank that is accessible via the network.

### BACKGROUND OF THE INVENTION

1. Field of the Invention

The invention is directed to a method and a system for acquiring at least one measured quantity for diagnosis or evaluation of the pathogenesis of a central nervous system disorder of a person.

2. Description of the Prior Art

For diseases of the central nervous system, comprehension, time and place orientation, memory power as well as voluntary motor functions of a person represent important measured quantities for diagnosis, for evaluating the pathogenesis of a person, or for evaluating the effectiveness of a therapy. The acquisition of such measured quantities currently ensues with specifically developed tests that are implemented on local systems usually arranged in medical facilities. The NC SYS company—Neuro Cognitive Systems—, for example, offers an integrated therapy system “IST”) for cognitive and neurological examinations of persons (available at http://www.ncsys.de/).

A disadvantage of this type of acquisition of measured quantities for the diagnosis of functional capacity disorders of the brain of a person, for evaluating the course of a central nervous system disorder of a person, is that the person must visit a medical facility for the implementation of a test. This involves a large time expenditure for the person and a high administrative outlay for the medical facility with respect to setting up an appointment and the usage of the system.

### SUMMARY OF THE INVENTION

An object of the invention is to provide a method and a system of the type initially described wherein the acquisition of a measured quantity can be more comfortably implemented for the person and more economically for the medical facility.

This object is inventively achieved in a method and system wherein at least one measured quantity for diagnosis or evaluating the pathogenesis of a central nervous system disorder of a person is acquired while the person, using a computer, plays a game offered on a central computer via a communication network, and the measured quantity is related to play of the game, such as a game result that can be influenced by the person, or an indicator of the game-playing behavior of the person. The measured quantity is stored for later evaluation in a central data bank. A person for whom a diagnosis is to be prepared or a person for whom the pathogenesis of a central nervous system disorder is to be evaluated no longer must visit a medical facility. On the contrary, the acquisition of measured quantities that are of assistance for the diagnosis or for the review of the condition of a person can be implemented in a comfortable way using a personal computer connected to the central computer via a communication network, which is preferably the Internet, whereby the person can be at home or at some other, nearly arbitrary location. An advantage for a medical facility is that persons or patients can initiate such checks on their own with an Internet-compatible computer, so that tests to be implemented in the medical facility and the administrative outlay connected therewith are eliminated. Moreover, persons can play the games simultaneously regardless of the time of day without the provision of special measures by the medical facility and without burdening the operation of the medical facility.

A further advantage of the invention is comprised therein that no specific test procedures are employed for the acquisition of the measured quantity. On the contrary, the determination ensues by means of a computer game. According to one version of the invention, this is a matter of a commercially obtainable computer game, for example memory, Mindman, turkey hunt or some other known computer game. In this way, persons and patients are motivated to voluntarily provide the evaluation data and to do so as regularly as possible.

In an embodiment of the measured quantity acquired during the course of the game is one or more of the best game result of the person, the average time duration between two game inputs of the person, the number of incorrect attempts that the person requires on average until an objective required in the game has been solved, and the game level the person achieves. Such measured quantities thus can be acquired in a simple way. The game result that is achieved as well as the game level that is reached can be queried directly from the computer game that has been played, and this can occur after the completion of play. The determination of the average time duration between two game inputs of the person can be realized with a simple time acquisition, with the time from one input until the next game input always being measured when the person undertakes a game input and, subsequently, the average time duration is calculated. The average number of incorrect attempts likewise can be obtained with a simple count with subsequent calculation of the average of those inputs that did not lead to the solution of a task or a sub-task. Which measured quantities are acquired also is dependent on the respective game. When, for example, a person plays the game “memory”, the average number of incorrect attempts until two coinciding images have been found is acquired in addition to the average time duration that the person needs between two game inputs, for example uncovering cards (move speed of the person). Further, the game level, i.e. the difficulty degree, can be acquired, this being composed of the degree of complexity of the turn-over pattern. In the simplest embodiment of the invention, only the best result of the game that has been achieved, referred to as the high score, is acquired as measured quantity and stored.

In order to be able to uniquely allocate the game results to only one person and to prevent a misuse of the system, in versions of the invention the identity of the person currently playing is determined. This can be achieved by assigning a login to every person using the system, the login including a user name and a password. Further identification possibilities for a person are assigning an IP address or using a chip card, that latter assumes that a chip card reader is connected to the computer at which the person will play the game. Another identification possibility is the acquisition of at least one characteristic physical feature of a person for the identification of the person. For example, the acquisition of a fingerprint of the person with a fingerprint sensor as well as iris monitoring can be used for this purpose.

In an embodiment of the invention, epidemiological data of the person are preferably acquired before the beginning of a game. Such data include, for example, the age, sex, state of health of the person, or medications taken by the person. Such data usually have to be acquired only when the person plays a game for the first time for the acquisition of measured quantity for diagnosis or for evaluation of the pathogenesis of a disorder of the central nervous system, or when significant changes have occurred in the epidemiological data, for example a change in the taking of medications.

In a preferred embodiment of the invention, the measured quantities of a person acquired during a number of sessions of a specific game are automatically statistically interpreted, preferably with an analysis device. The analysis device can be the central computer itself or can be a computer dedicated for this purpose that statistically edits the acquired data for the person and stores the statistical analysis in the data bank. For example, dependent on the number of game performances the curve of the high scores can thereby be determined, with the number of game performances entered on the abscissa and the high scores entered on the ordinate. The skill levels that have been reached or the average time duration between two game inputs likewise can be presented in curves in this way, i.e. dependent on the game sessions.

According to a further embodiment of the invention, a group of an optimum number of persons is formed on the basis of the epidemiological data the persons, and comparison values for diagnosis or for evaluating the pathogenesis of a central nervous system disorder are automatically generated on the basis of the statistical analyses of the measured quantities of the persons from the group. The automatic generation of comparison values can, for example, ensue by statistical analyses of the game results and game-related measured quantities of central nervous system disorder, persons who have no or at least not a comparable central nervous system disorder, as well as substantially identical basic data with respect to age, sex, education, profession, etc. The comparison results are determined by forming averages, correlation algorithms or by applying other, suitable mathematical methods. An initial diagnosis or an initial evaluation of the pathogenesis of the person under observation is possible using the comparison values, based on game results and game-related measured quantities of those in a group to which the person under observation would otherwise belong, but for the presence of the central nervous system disorder.

In another embodiment of the invention, the person-specific comparison values for the evaluation of the pathogenesis of a central nervous system disorder are automatically generated from the statistical analysis of the measured quantities of the person. The automatic generation of person-specific comparison values, i.e. individual comparison values, ensues by the game results and game-related measured quantities of the respective person being acquired over a specific time span, followed by a statistical analysis thereof from which one or more comparison values for the respective person are determined, for example by forming averages or with some other suitable mathematical methods. The comparison values can be adapted dependent on the pathogenesis of the person. When, for example, a person having a central nervous system disorder demonstrates continuously improved game results, this person's individual comparison values are automatically adapted, for example in the direction of the comparison values of a group that has healthy players, i.e. persons without a central nervous system disorder.

In aversion of the invention, the best result or results that a person has when playing a game are stored and are made reavailable for comparison purposes the next time the game is played. In this way, a stimulus is created for the person to re-play the game anew and to improve the results previously achieved.

In a preferred embodiment of the invention provides that the measured quantities of a person that are acquired, and possibly statistically analyzed, can be called from the databank by a physician via a communication network. A physician treating a person thus has access to the original measured quantities in a simple way, and to the measured quantities that have already been statistically analyzed (if this is done) in order to prepare a diagnosis about the condition of the pathogenesis on the basis of the measured quantities, and to initiate suitable therapy if necessary. Additionally, the physician can continuously stay informed about the pathogenesis and accompanying deterioration or improvement in the health of the person, and can initiate measures as needed.

In a version of the invention, a discussion session, known as a chat, is made available for physicians or therapists on the central computer, the therapists and/or physicians communicating via this medium and being able to exchange diagnostic or therapeutic questions with one another in a simple way.

### DESCRIPTION OF THE DRAWINGS

FIG. 1 illustrates a network structure of a system for acquiring at least one measured quantity for diagnosis or evaluation of the pathogenesis of a central nervous system disorder of a person in accordance with the invention.

FIG. 2 illustrates two statistical analyses of acquired measured quantities in accordance with the invention.

### DESCRIPTION OF THE PREFERRED EMBODIMENTS

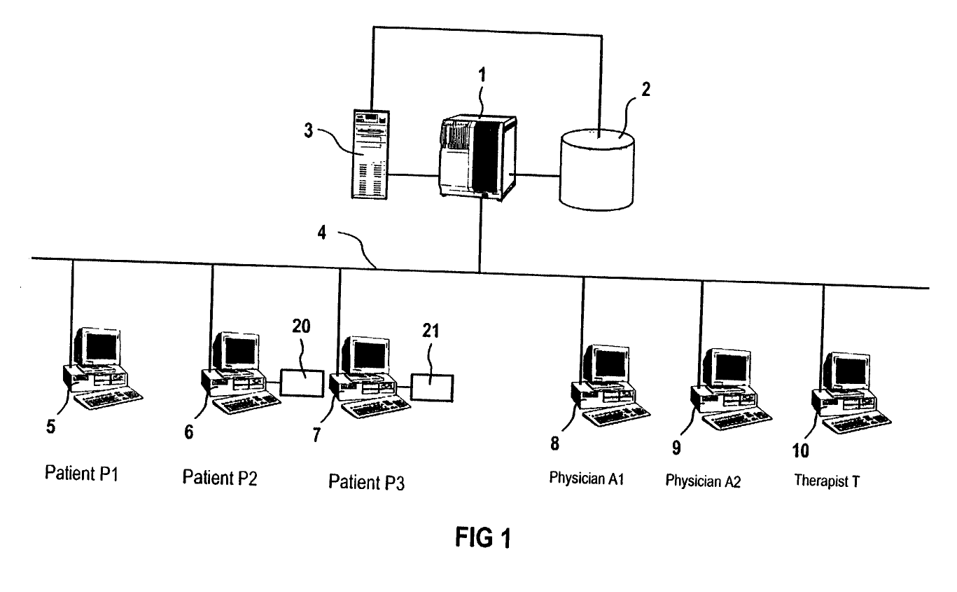

In an exemplary embodiment, the system shown in FIG. 1 for acquiring at least one measured quantity for diagnosis or for evaluating the pathogenesis of a central nervous system disorder of a person that, accordingly, serves for checking the cognitive and coordinative functions of the person, has a server to which a central databank 2 and an analysis device 3, which is a computer, are connected. The server 1 itself is connected via a known interface to a public communication network 4, which is the Internet in the exemplary embodiment. In the exemplary embodiment, moreover, three Internet-compatible computers 5 through 7 arranged at different locations and respectively allocated to patients P1 through P3 are also connected to the communication network 4. Three other Internet-compatible computers 8 through 10 that are respectively allocated to physicians A1, A2, and to a therapist T are likewise connected to the communication network 4. Accordingly, communication is possible between the computers 5 through 10 and the server 1, so that information and programs made available on the server 1 can be called in a known way given knowledge of their Internet addresses, proceeding from each of the computers 5 through 10.

In the exemplary embodiment, the server 1 makes a number of commercially available computer games available, that the patients P1 through P3 can play on their computers 5 through 7, via the communication network 4. The games that are made available are known computer games such as mindman, turkey hunt or memory. The games, however, are provided with an additional software component, so that the inputs of a player, i.e. of one of the patients P1 through P3, that relate to the game and may influence the course of the game can be registered and measured quantities derived therefrom can be stored in the databank 2. A finding thus can be made on the basis of the measured quantities as to whether the player has a central nervous system disorder. Further, the status of the disorder as well as the pathogenesis thereof can be identified.

For acquiring measured quantities, the person P1 calls the Internet address of the home page of the computer games made available on the server 1 using a software in the form of a known network browser, for example the program “Netscape”. On the occasion of the initial call, the patient P1 arrives in an input mask for registering the patient into which the patient P1 must enter epidemiological data. The epidemiological data include, for example, one or more of the age, sex, education, profession, and state of health of the patient P1. Further, details about medications taken by the patient P1 or the course of a central nervous system disorder of the patient P1 may be required. In such a registration of the patient P1, a login for the patient P1 including a user name and a password is assigned with which the patient P1 can login in the future for playing games at the server 1. The user name as well as the password usually can be freely selected by the patient P1. When the patient, after calling the home page, has logged in with his or her user name and password, the patient can select a game from a number of computer games that the patient would like to play for checking the patient's condition. The selection and the implementation of the game ensue in a known way. The measured quantities about the game behavior of the patient are acquired in a manner that is invisible to the patient P1, these measured quantities providing information about a central nervous system disorder, or the pathogenesis of such a disorder. When, for example, the patient P1 plays the game “memory”, then, for example, one or more the average time duration between two moves of the patient P1 is acquired by time measurement, the average number of incorrect attempts until two coinciding images have been found is acquired by counting and the game level that has been reached, the degree of difficulty that has been reached and that is revealed in the complexity of the turn-over pattern, is/are acquired with an additional software component. These measured quantities acquired during the playing of the game, finally, are stored in the databank 2 under the identification of the patient P1. With a corresponding access authorization to the databank 2 and to the measured quantities of the patient P1, a physician treating the patient P1 or a therapist can call the measured quantities of the patient P1 from the databank 2 for diagnostic purposes and for setting up a therapy. For example, the physician A1, using the communication network 4 and the server 1, can call the measured quantities of the patient P1 acquired during the course of the game with his computer 8.

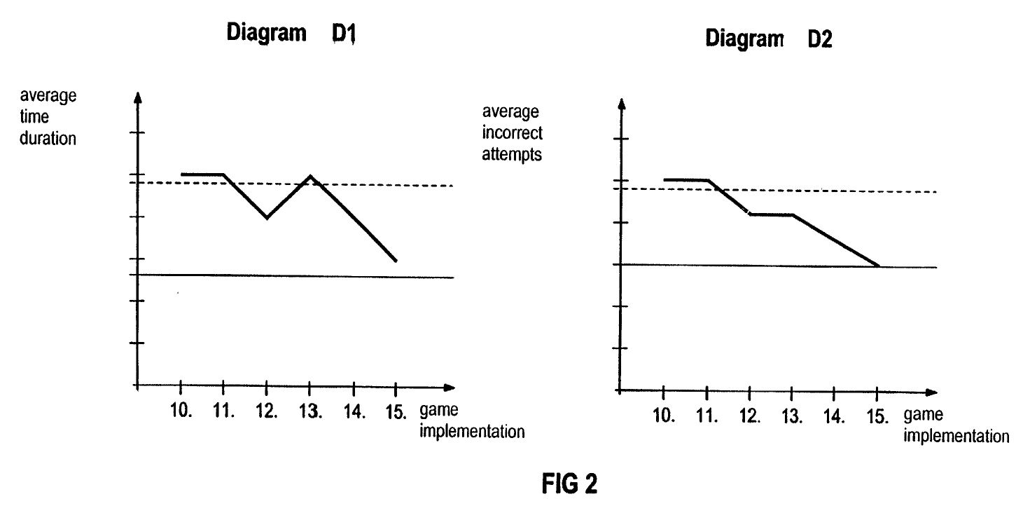

In the exemplary embodiment, however, not only are the original (raw) measured quantities that were acquired during the course of the playing of the game stored in the databank 2; rather, but also analysis results of these measured quantities, automatically analyzed with the analysis device 3, are stored therein. The raw data, for example, can be statistically analyzed. FIG. 2 shows two statistical analyses for the person P1 who has already played the game “memory” a number of times. In the first diagram D1, thus, the time duration that the person P1 requires on average between the uncovering of two cards per game is entered over the multiple playing sessions of the game. In the second diagram D2, the number of incorrect attempts that the person P1 had on average until two cards that belong together were found is entered over the multiple playing sessions of the game.

In addition, these statistical measured results can be placed into relationship with prescribed comparison values by the analysis device 3. Such comparison values, however, also can be automatically generated with the analysis device 3. Comparison values can be automatically generated from measured quantities or game results of a group of players who have comparable prerequisites in view of education as well as age and sex and are preferably healthy, i.e. do not have any disorders of the central nervous system. These comparison values are automatically generated, for example, by forming averages of measured quantities acquired during the games, for instance the average time duration lying between two game inputs, the number of incorrect attempts that a person requires on average until solving a task setup in a game, the game level achieved or the best game result of a person or on the basis of some other mathematical processing of the measured quantities acquired during the games. When, for example, statistical analyses for the game “memory” are present for each person of a group of persons in the form of the diagrams D1 and D2, then, for example by forming averages, an average time duration between two game moves or an average plurality of incorrect attempts can be determined via the statistical analyses of the persons. Two solid lines that represent these comparison values from the group are entered in diagrams D1 and D2 in FIG. 2.

There is also the possibility of automatically generating person-specific comparison values. The determination of such person-specific comparison values preferably ensues by acquiring measured quantities of the respective patient over a specific time span, taking the changes in the game results of the patient over time into consideration. Comparison values for individual patients can be defined automatically in this way and can be compared to the currently achieved measured quantities of the patients. In the exemplary embodiment, game results of the person P1 from the game “memory” are already known from the game implementations 1 through 9 that were analyzed before the game implementations 10 through 15 covered in the diagrams D1 and D2. The person-specific comparison values of the patient P1 entered as broken lines in the diagrams D1 and D2 were determined from these results of the patient P1 by forming averages in the present case. It can be seen therefrom that the game results of the patient P1 in the implementations of the game “memory” documented in the diagrams D1 and D2 have already improved compared to the earlier game results contained in the person-specific comparison values.

Overall, thus, it can be derived from the diagrams D1 and D2 that the patient P1 already has made progress but still falls short of a comparison group in view of the patient's game results.

A physician A1, A2 or therapist T treating a patient P1 through P3, given appropriate authorization, can access the data stored in the databank 2 via the communication network 4 for diagnosis and the selection of a suitable therapy.

In order to create a stimulus for the patients P1 through P3 to repeatedly play the games and thus to allow continuous identification of changes in the central nervous system disorders of the patients P1 through P3, the game results—preferably only the best results—for the patients P1 through P3 are retained and displayed when the game is played again. There is a motivation for the patients P1 through P3 to improve their previous best results, referred to as the high scores.

Further, the central computer allows a discussion in the form of a chat, via which the physicians A1, A2 and therapist T can communicate with one another on line and discuss diagnostic and/or therapeutic questions as well as select suitable games for determining relevant data.

In the way described for the patient P1, the patients P2 and P3 or other patients as well as, in particular, healthy persons can also play the games made available on the server 1 via the communication network 4, whereby measured quantities for diagnosis or for evaluating the pathogenesis of a central nervous system disorder of a person are acquired and are stored in the databank 2 as original measured quantities or as measured quantities statistically analyzed by the analysis device 3. Whereas the measured quantities of patients acquired during the games serve mainly for diagnosis and for selecting therapies as well as for determining person-specific comparison values, the measured quantities of healthy persons acquired during the games are mainly employed for the described, automatic generation of group comparison data.

The identity of the playing persons need not necessarily ensue by means of a user name and a password. Further possibilities for identifying persons are the employment of chip cards or in the identification of body-specific features. For employment of a chip card on which the identification of person is stored, however, a chip card reader that is connected to the respectively employed computer. Such a chip card reader 20 is connected to the computer 6 of the patient P2 in FIG. 1. The identification of a person via a body-specific feature can, for example, ensue with a fingerprint sensor, as schematically shown in FIG. 1, that is connected to the computer 7 of the patient P3.

Standard home PCs preferably are used as the computers for patients, persons, physicians and therapists, as well as, laptops and other computer devices that can be operated in network-independent fashion as well.

The type of measured quantities that are acquired during a game are dependent on the respective game, i.e. dependent on what can be acquired at all.

The communication network, moreover, need not necessarily be the Internet, but also may be an Intranet or some other computer network.

Although modifications and changes may be suggested by those skilled in the art, it is in the intention of the inventors to embody within the patent warranted hereon all changes and modifications as reasonably and properly come within the scope of their contribution to the art.

We claim as our invention:

1. A method for acquiring a measured quantity for diagnosis or evaluation of the pathogenesis of a central nervous system disorder of a patient, comprising the steps of:
connecting at least one computer to a central computer via a communication network and offering a game, playable on said computer by a person operating said computer, from said central computer to said computer via said network;
acquiring a measured quantity relating to play of said game by said person at said computer selected from the group consisting of a game result influenced by said person and an indicator of play behavior of said person in playing said game;
transmitting said measured quantity from said computer to said central computer via said network and storing said measured quantity at a central data bank connected to said central computer; and
accessing said measured quantity from said data bank and using said measured quantity to obtain an analysis result selected from the group consisting of a diagnosis of a central nervous system disorder of a patient and an evaluation of the pathogenesis of a central nervous system disorder of a patient.

2. A method as claimed in claim 1 wherein said person is said patient.

3. A method as claimed in claim 2 comprising the additional steps of:
acquiring a further measured quantity, corresponding to the measured quantity acquired from said patient, from a further person playing said game who does not have said central nervous system disorder; and
in obtaining said analysis result, using said further measured quantity as a comparison value for comparison with the measured quantity acquired from said patient.

4. A method as claimed in claim 1 comprising offering a commercially-available computer game from said central computer as said game.

5. A method as claimed in claim 1 wherein said measured quantity is selected from the group consisting of a best game result of said person, an average time duration between two game inputs by said person, an average number of incorrect attempts required by said person until an objective required in said game is achieved, and a game level achieved by said person.

6. A method as claimed in claim 1 comprising the additional steps of:
acquiring the identity of said person via said computer;
transmitting said identity of said person via said network to said central data bank together with said measured quantity from said person; and
storing said identity in said central data bank allocated to said measured quantity from said person.

7. A method as claimed in claim 1 comprising the additional steps of:
acquiring epidemiological data from said person via said computer, selected from the group consisting of the age of said person, the sex of said person, a state of health of said person and medications being taken by said person;
transmitting said epidemiological data from said computer via said network together with said measured quantity acquired from said person; and
storing said epidemiological data in said central data bank allocated to said measured quantity acquired from said person.

8. A method as claimed in claim 1 wherein said person is said patient and wherein said patient repeatedly plays said game in a plurality of play sessions, including previous sessions and a current session, and wherein said measured quantity is acquired from said patient during each of said plurality of play sessions, thereby generating a plurality of measured quantities which are stored in said central data bank, and wherein the step of obtaining an analysis result comprises automatically statistically analyzing said plurality of measured quantities from said previous sessions to obtain a patient-specific comparison value, and comparing said comparison value to the measured quantity acquired in said current session.

9. A method as claimed in claim 8 wherein said measured quantity is said game result, and comprising the additional steps of:
storing a best game result of said patient from among said plurality of play sessions; and
making said best game result available to said patient via said network and said computer at a subsequent play session.

10. A method as claimed in claim 1 wherein said person is said patient, and comprising the additional steps of:
acquiring a plurality of further measured quantities respectively from a plurality of further persons playing said game;
from said patient and from said further persons, acquiring respective sets of epidemiological data via said computer, said epidemiological data including, for each of said patient and said further persons, age, sex, state of health and medications being taken;
transmitting said sets of epidemiological data together with said plurality of measured quantities to said central data bank and storing the plurality of measured quantities in said central data bank respectively associated with the sets of epidemiological data; and
in obtaining said analysis result, forming at least one group from among said further persons comprised of persons having similar epidemiological data, and automatically statistically analyzing the respective measured quantities of the persons comprising said at least one group to obtain a comparison value, and comparing said measured quantity acquired from said patient to said comparison value.

11. A method as claimed in claim 1 comprising connecting a further computer to said central computer via said network and allowing access of said measured quantity and said analysis result from said central data bank via said network.

12. A method as claimed in claim 1 comprising connecting a plurality of further computers to said central computer via said network and allowing access to said measured quantity and said analysis result by each of said further computers via said network, and further comprising said central computer making a chat format available to said plurality of further computers via said network for a discussion relating to said central nervous system disorder of said patient.

13. A system for acquiring a measured quantity for diagnosis or evaluation of the pathogenesis of a central nervous system disorder of a patient, comprising:
at least one computer and a central computer connected via a communication network, said central computer offering a game, playable on said computer by a person operating said at least one computer, from said central computer to said at least one computer via said network;
said at least one computer acquiring a measured quantity relating to play of said game by said person at said at least one computer selected from the group consisting of a game result influenced by said person and an indicator of play behavior of said person in playing said game;
said at least one computer transmitting said measured quantity from said at least one computer to said central computer via said network;
a central data bank connected to said central computer in which said measured quantity is stored; and
said central computer accessing said measured quantity from said data bank and using said measured quantity to obtain an analysis result selected from the group consisting of a diagnosis of a central nervous system disorder of a patient and an evaluation of the pathogenesis of a central nervous system disorder of a patient.

14. A system as claimed in claim 13 wherein said person is said patient.

15. A system as claimed in claim 14 wherein said at least one computer acquires a further measured quantity, corresponding to the measured quantity acquired from said patient, from a further person playing said game who does not have said central nervous system disorder, and wherein in obtaining said analysis result, said central computer uses said further measured quantity as a comparison value for comparison with the measured quantity acquired from said patient.

16. A system as claimed in claim 13 wherein said central computer offers a commercially-available computer game as said game.

17. A system as claimed in claim 13 wherein said measured quantity is selected from the group consisting of a best game result of said person, an average time duration between two game inputs by said person, an average number of incorrect attempts required by said person until an objective required in said game is achieved, and a game level achieved by said person.

18. A system as claimed in claim 13 wherein said at least one computer acquires the identity of said person and transmits said identity of said person via said network to said central data bank together with said measured quantity from said person, wherein said central data bank stores said identity allocated to said measured quantity from said person.

19. A system as claimed in claim 13 wherein said at least one computer acquires epidemiological data from said person, selected from the group consisting of the age of said person, the sex of said person, a state of health of said person and medications being taken by said person and transmits said epidemiological data from said computer via said network together with said measured quantity acquired from said person, and wherein said control data bank stores said epidemiological data allocated to said measured quantity acquired from said person.

20. A system as claimed in claim 13 wherein said person is said patient and wherein said patient repeatedly plays said game in a plurality of play sessions including previous sessions and a current session, and wherein said at least one computer acquires said measured quantity from said patient during each of said plurality of play sessions, thereby generating a plurality of measured quantities which are transmitted to and stored in said central data bank, and wherein said central computer obtains said analysis result by automatically statistically analyzing said plurality of measured quantities from said previous sessions to obtain a patient-specific comparison value and, compares said comparison value to the measured quantity acquired in said current session.

21. A system as claimed in claim 20 wherein said measured quantity is said game result, and wherein said central data bank stores a best game result of said patient from among said plurality of play sessions and makes said best game result available to said patient via said network and said computer at a subsequent play session.

22. A system as claimed in claim 13 wherein said person is said patient, and wherein said at least one computer acquires a plurality of further measured quantities respectively from a plurality of further persons playing said game and from said patient and from said further persons, acquires respective sets of epidemiological data, said epidemiological data including, for each of said patient and said further persons, age, sex, state of health and medications being taken, and wherein said at least one computer transmits said sets of epidemiological data together with said plurality of measured quantities to said central data bank and wherein said central data bank stores the plurality of measured quantities respectively associated with the sets of epidemiological data, and in obtaining said analysis result, forms at least one group from among said further persons comprised of persons having similar epidemiological data, and automatically statistically analyzes the respective measured quantities of the persons comprising said at least one group to obtain a comparison value and compares said comparison value to said measured quantity acquired from the patient.

23. A system as claimed in claim 13 comprising a further computer connected to said central computer via said network allowing access of said measured quantity and said analysis result from said central data bank via said network.

24. A system as claimed in claim 13 comprising a plurality of further computers connected to said central computer via said network allowing access to said measured quantity and said analysis result via each of said further computers via said network, and wherein said central computer makes a chat format available to said plurality of further computers via said network for a discussion relating to said central nervous system disorder of said patient.
